(19) United States
(12) Patent Application Publication
(10) Pub. No.: US 2026/0096558 A1
(43) Pub. Date: Apr. 9, 2026

(54) THE USE OF PHYTIC ACID CHELATOR FOR MICROBIAL RAPID KILL

(71) Applicant: Henkel AG & Co. KGaA, Düsseldorf (DE)

(72) Inventors: Joseph D. Dahlmeyer, JR., East Haven, CT (US); Gregory A. Cole, Stamford, CT (US)

(21) Appl. No.: 18/905,267

(22) Filed: Oct. 3, 2024

(51) Int. Cl. A01N 57/12; A01N 59/00; A01P 1/00; A61K 8/27; A61K 8/55; A61Q 19/10; C11D 3/02; C11D 3/36; C11D 3/48

(52) U.S. Cl. CPC A01N 57/12; A01N 59/00; A01P 1/00; A61K 8/27; A61K 8/55; A61Q 19/10; C11D 3/02; C11D 3/362; C11D 3/48

(57) ABSTRACT

Disclosed is the use of phytic acid and a zinc compound in an antimicrobial composition for microbial rapid kill. The antimicrobial composition finds particular application in liquid antimicrobial compositions for consumer products.

### TECHNICAL FIELD

The present disclosure relates to antimicrobial compositions and their uses, in particular antimicrobial compositions comprising phytic acid and use in methods of disinfection.

### BACKGROUND

Antibacterial personal care compositions such as antibacterial cleansing compositions are known in the art. These are typically used to cleanse the skin and to destroy bacteria and other microorganisms present on the skin, especially on the hands, arms, and face of the user. Various classes of antibacterial agents have been used in antibacterial cleansing compositions including quaternary ammonium compounds, which can be used in combination with a chelating agent. Known chelating agents include ethylenediaminetetraacetic acid (EDTA) and ethylenediaminedisuccinic acid (EDDS). A disadvantage of EDTA is that it is not biodegradable.

There remains a need in the art for antimicrobial compositions that have high efficacy in killing bacteria or inhibiting bacteria growth on the skin, while also containing naturally sourced, biodegradable, and environmentally safe ingredients.

### SUMMARY

Disclosed, in various embodiments, are antimicrobial compositions comprising phytic acid; a zinc compound; and a liquid carrier.

In another embodiment, a method of disinfecting comprising, contacting a surface with an antimicrobial composition comprising phytic acid and a zinc compound.

In another embodiment, a method of washing the skin of a human, comprising washing the skin with an antimicrobial cleaning composition; and rinsing the antimicrobial cleaning composition from the skin; wherein the antimicrobial cleaning composition comprises phytic acid, a zinc compound, a surfactant, and a liquid carrier.

These and other features and characteristics are more particularly described below.

### BRIEF DESCRIPTION OF THE DRAWINGS

Referring now to the figures, which are exemplary embodiments, and wherein the like elements are numbered alike:

FIG. 1 illustrates a Ternary Plot showing a Mixture Design of Experiment design for combinations of benzalkonium chloride/phytic acid/ZnSO₄ in liquid hand soap formulations screened against three bacteria species for rapid time kill at 30 seconds according to ASTM E 2783.

FIG. 2 illustrates a mixture profiler of efficacy against E. coli where a minimum time kill efficacy threshold is set at 99.9%.

FIG. 3 illustrates a mixture profiler of efficacy against P. aeruginosa where a minimum time kill efficacy threshold is set at 99.9%.

FIG. 4 illustrates a mixture profiler of efficacy against S. aureus where a minimum time kill efficacy threshold is set at 99.9%.

FIG. 5 illustrates a prediction profiler created from a DOE to identify compositions which maximize the desirability of the overall model.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Disclosed are antimicrobial compositions and their uses, in particular antimicrobial compositions comprising phytic acid and their use in methods of disinfection. In general, the antimicrobial compositions comprise an effective amount of phytic acid and a carrier. The antimicrobial compositions further comprise a zinc compound which can provide a synergistic benefit when used in combination with the phytic acid. The compositions are highly efficacious, exhibiting microbial rapid kill against a variety of microorganisms, both Gram-negative and Gram-positive.

Phytic acid is a chelating agent that is sourced naturally and is readily biodegradable and environmentally friendly. Without being bound by theory, it is believed that phytic acid can act as a chelator at or around skin pH (4.7-5.5) and will remove calcium and/or magnesium from the bacteria cell membrane. As calcium and magnesium are structural stabilizing atoms in the cell membrane, it is hypothesized that the removal of such divalent cations will destabilize the bacteria membrane, resulting in weak spots. These weak spots may result in the introduction of external materials or the leakage of internal cellular components. In either case, it is hypothesized that this destabilization will result in rapid bacteria death. There are kinetic factors to consider in the above description, as the chelating/destabilizing species must be able to rapidly diffuse to the local regions of the bacteria cell membranes that contain calcium or magnesium and must be able to rapidly extract these cations. Additionally, there is expected to be a pH dependence for this mechanism. The molecular structure of phytic acid and the pKa values for the six phosphate groups of phytic acid are given below:

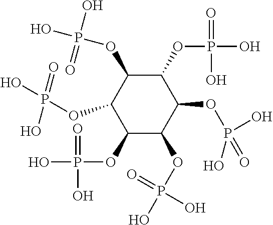 [SMILES: O=P(O)(O)O[C@H]1[C@H](OP(=O)(O)O)[C@@H](OP(=O)(O)O)[C@H](OP(=O)(O)O)[C@@H](OP(=O)(O)O)[C@H]1OP(=O)(O)O]

or 3D configuration

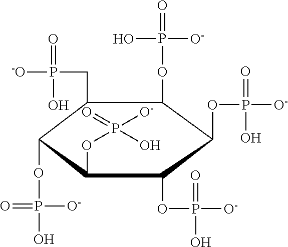

(2,3,4,5,6-pentaphosphonooxycyclohexyl) dihydrogen phosphate; myo-inositol, 1,2,3,4,5,6-hexakis (dihydrogen phosphate); CAS No. 83-86-3, free phytic acid.
6 protons: pKa 1.1-2.1
3 protons: pKa 5.7-7.6
3 protons: pKa 10.0-12.0

The pH for an antibacterial hand soap is about 4.7 to about 5.5. Therefore, for use in liquid hand soap it is expected that only 6 protons (one from each phosphate group) will be removed from the molecule at the hand wash pH, as shown in the 3D configuration above. In order to effectively extract divalent calcium or magnesium, it would be expected that two phosphate groups would be required to simultaneously coordinate with the metal centers. Therefore, the extent of the effectiveness of phytic acid in extracting divalent cations is unknown and may be limited by steric hinderances of adjacent phosphate groups and limited three-dimensional rotation due to rigidity in the six-membered carbon ring.

Molecules such as ethylenediaminetetraacetic acid (EDTA) and ethylenediaminedisuccinic acid (EDDS) are known to effectively coordinate divalent cations. This is due to their respective pKa values at a pH of about 4.7 to about 5.5 and the benefits of having the carboxylate groups connected by C—N—C bonds that can rotate easily in three-dimensional space which allows for optimal binding proximity.

Antimicrobial compositions described herein comprise an effective amount of phytic acid and a zinc compound which provide a surprisingly synergistic effect for microbial rapid kill against several species of microorganisms, both Gram-negative and Gram-positive. The composition further comprises a carrier, specifically a liquid carrier.

The phytic acid component can be provided in solution form in water, for example a 50% solution in water. Alternatively, the phytic acid can be provide in a solution form in water and a lower alcohol. The phytic acid component used to prepare the antimicrobial compositions described herein can be free phytic acid or a phytic acid salt, for example an alkali salt, specifically sodium or potassium, or an alkaline earth metal salt, such as calcium.

Zinc compounds provide zinc ions in the compositions. Zinc ions have biocidal properties. Zinc is provided as zinc sulfate in the Examples herein, however, other sources of zinc ions may be substituted with predictable effects. For example, zinc may be provided as a complexed species such as zinc picolinate, zinc citrate, zinc acetate, zinc glycerate, zinc gluconate, or zinc monomethionine. Zinc may be provided in other forms for example, zinc chloride, zinc iodide, zinc oxide, and the like.

The quantity of phytic acid and zinc compound in the antimicrobial composition can depend on the end use of the composition and the types of additional ingredients.

In an antimicrobial cleaning composition, such as a liquid hand soap, the phytic acid can be present in an amount of about 0.001 weight percent (wt %) to about 2.0 wt % based on the total weight of the antimicrobial cleaning composition, specifically about 0.005 wt % to about 1.0 wt %, more specifically about 0.01 wt % to about 0.5 wt %, yet more specifically about 0.025 to about 0.25 wt %, and still yet more specifically about 0.045 to about 0.1 wt %.

In an antimicrobial cleaning composition, such as a liquid hand soap, the zinc compound may be present in an amount of about 0.001 wt % to about 2.0 wt % based on the total weight of the antimicrobial cleaning composition, specifically about 0.005 wt % to about 1.0 wt %, more specifically about 0.01 wt % to about 0.5 wt %, yet more specifically about 0.025 to about 0.25 wt %, and still yet more specifically about 0.045 to about 0.1 wt %.

The phytic acid and the zinc compound can be present in the antimicrobial composition and antimicrobial cleaning composition in a weight ratio of about 75:25 to about 25:75; specifically about 70:30 to about 30:70; more specifically about 65:35 to about 35:65; still more specifically about 60:40 to about 40:60; and about 50:50.

The liquid carrier of the antimicrobial composition can include polar solvents such as water, lower alcohols such as ethanol, propanol, isopropanol, propylene glycol, glycerin, and the like, or a combination thereof. In certain embodiments, the liquid carrier is water.

The quantity of liquid carrier in the antimicrobial composition can depend on the end use of the composition and the types of additional ingredients. In an embodiment, the quantity of liquid carrier, including an aqueous carrier, can be about 40 wt % or greater based on the total weight of the antimicrobial composition, specifically about 50 wt % or greater, and more specifically about 60 wt % or greater. In an antimicrobial cleaning composition, such as a liquid hand soap, the quantity of liquid carrier, specifically water, can be about 65 wt % or greater based on the total weight of the antimicrobial cleaning composition, specifically about 70 wt % or greater, and more specifically about 75 wt % or greater.

The antimicrobial compositions can be formulated into antimicrobial cleaning compositions. In an embodiment, the antimicrobial cleaning compositions can be for use on the body including liquid hand soap, body wash, facial cleansers, disinfecting wipes, hand sanitizer solutions and the like; formulated appropriately for more robust cleaning (e.g. liquid hand soap) or for milder cleansing (e.g. body wash). In an alternative embodiment, the antimicrobial composition can be formulated as a concentrate of an antimicrobial cleaning composition, to be diluted with water to result in the antimicrobial cleaning composition. Examples of suitable weight ratios of concentrate to water for dilution can be about 1:2 to about 1:4, specifically about 1:3.

The antimicrobial compositions can have a pH of about 3.5 to about 7.6, specifically about 4.0 to about 7.0, more specifically about 4.5 to about 6.0, yet more specifically about 4.6 to about 5.5, and still yet more specifically about 4.7 to about 5.0.

The antimicrobial compositions may also include an additional ingredient such as an additional biocide, a buffer, a colorant/dye, a fragrance, a humectant, an emollient, a natural plant oil or extract, a pH adjusting agent, a preservative, a salt, a solvent, a surfactant, a thickener, or a combination thereof. The additional ingredient can be a benefit or care agent, or other conventional ingredient used in cleaning formulations and cosmetic cleaning products.

The antimicrobial compositions may optionally further comprise an additional biocide, such as a cationic biocide. In some examples, these are quaternary amine biocides, for example: benzalkonium chloride (BKC), benzethonium chloride (BZC), cetrimonium chloride (CC), cetrimonium bromide (CTAB), or a combination thereof. More complex structures, such as bisbiguanides, e.g., chlorhexidine and polymeric biguanides, e.g., polyhexamethylene biguanide may be used as an additional biocide.

Suitable buffers include citric acid/citrate.

Suitable pH adjusting agents include mineral acids, organic acids such as adipic acid, acetic acid, azelaic acid, citric acid, glutaric acid, maleic acid, malic acid, oxalic acid, pimelic acid, sebacic acid, suberic acid, succinic acid, and the like.

Suitable preservatives include benzoic acid and salts thereof, e.g. sodium benzoate, sorbic acid, and the like.

Suitable humectants include aloe, glycerin (glycerol), butylene glycol, 1,4-dihydroxyhexane, dipropylene glycol, glycolic acid, 1,2,6-hexanetriol, hexylene glycol, hyaluronic acid or a salt thereof, lactic acid, propanediol, propylene glycol, sorbitol, triethylene glycol, a polyethylene glycol, a urea, or a combination thereof. When used in a cleaning composition, the humectant can be present in an amount of about 0.1 to about 20 wt % based upon the total weight of the antimicrobial cleaning composition, specifically about 1 to about 10 wt % and more specifically about 2 to about 5 wt %.

Suitable emollients for use in a cleaning composition include lipids and oils conventionally used in cosmetic applications, such as cocoa butter, shea butter, isopropyl palmitate, lineolic acid, and the like.

Antimicrobial cleaning compositions described herein further comprise a surfactant. Suitable surfactants can include anionic, amphoteric, nonionic, cationic surfactant, or a combination thereof. The type and amount of surfactant present in the composition is related to the identity of the surfactant or combination thereof, and the end use of the composition.

In an embodiment, the surfactant is cationic surfactant. Useful cationic surfactants include those having a structural formula

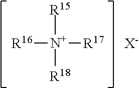

wherein R¹⁵ is an alkyl group having about 12 to about 30 carbon atoms, or an aromatic, aryl, or alkaryl group having about 12 to about 30 carbon atoms; each R¹⁶, R¹⁷, and R¹⁸, independently is hydrogen, an alkyl group having 1 to about 22 carbon atoms, or an aromatic, aryl, or alkaryl group having from about 12 to about 22 carbon atoms; and X is a compatible anion, specifically chloride, bromide, iodide, acetate, phosphate, nitrate, sulfate, methyl sulfate, ethyl sulfate, tosylate, lactate, citrate, glycolate, or a combination thereof. Additionally, the alkyl groups of R¹⁵, R¹⁶, R¹⁷, and R¹⁸ also can contain ester and/or ether linkages, or hydroxy or amino group substituents (e.g., the alkyl groups can contain polyethylene glycol and polypropylene glycol moieties).

In a specific embodiment R¹⁵ is an alkyl group having about 12 to about 22 carbon atoms; R¹⁶ is H or an alkyl group having 1 to about 22 carbon atoms; and R¹⁷ and R¹⁸, independently are H or an alkyl group having 1 to about 3 carbon atoms. More specifically R¹⁵ is an alkyl group having about 12 to about 22 carbon atoms, and R¹⁶, R¹⁷, and R¹⁸ are H or an alkyl group having 1 to about 3 carbon atoms.

Nonlimiting examples of quaternary ammonium salt cationic surfactants include cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, or a combination thereof.

In an embodiment the cationic surfactant is cetyl trimethyl ammonium chloride.

Additional quaternary ammonium salts include those where the C₁₂-C₃₀ alkyl carbon chain is derived from a tallow fatty acid or from a coconut fatty acid. The term “tallow” refers to an alkyl group derived from tallow fatty acids (usually hydrogenated tallow fatty acids), which generally has mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow) dimethyl ammonium chloride, di(hydrogenated tallow) dimethyl ammonium acetate, ditallow dipropyl ammonium phosphate, ditallow dimethyl ammonium nitrate, di(coconutalkyl) dimethyl ammonium chloride, di(coconutalkyl) dimethyl ammonium bromide, tallow ammonium chloride, coconut ammonium chloride, or a combination thereof.

Other useful cationic surfactants include amino-amides, wherein in the above structure R¹⁵ is R¹⁹CONH—(CH₂)ₙ—, wherein R¹⁹ is an alkyl group having about 12 to about 22 carbon atoms, and n is an integer of 2 to 6, specifically 2 to 4, and more specifically 2 to 3. Nonlimiting examples of these cationic surfactants include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof.

In an embodiment, the surfactant is an amphoteric surfactant such as an alkamine oxide, an alkylbetaine, an alkylamphoacetate, an alkylamphopropionate, an alkyliminopropionate, an amphoteric sulfonate, a sulfobetaine, or a combination thereof. In a further embodiment the alkylbetaine is an alkylamidobetaine, specifically cocamidopropyl betaine.

In various embodiments, particularly for antimicrobial cleaning compositions for use on the body, the surfactant can be an alkamine oxide surfactant. In an embodiment, the alkamine oxide surfactant can be present in an amount of about 0.1 to about 20 wt % based on the total weight of the antimicrobial cleaning composition, specifically about 0.5 to about 15 wt %, and more specifically about 1.0 to about 10 wt %.

Suitable alkamine oxide surfactants contain at least one long hydrocarbon chain containing at least eight carbon atoms. One class of alkamine oxides is the alkyl di(lower alkyl) amine oxides, wherein the alkyl group contains 8 to 22, and specifically about 10 to about 16, carbon atoms, and can be straight or branched chain, saturated or unsaturated. The lower alkyl groups contain 1 to 7 carbon atoms, and typically are methyl. Examples include lauryl dimethyl amine oxide, myristyl dimethyl amine oxide, dimethyl cocoamine oxide, dimethyl (hydrogenated tallow) amine oxide, myristyl/palmityl dimethyl amine oxide, myristyl/lauryl dimethyl amine oxide, cetyl dimethyl amine oxide, stearyl dimethyl amine oxide, myristyl/cetyl dimethyl amine oxide, and the like.

Another class of useful alkamine oxide surfactants includes alkyl di(hydroxy lower alkylamine oxides in which the alkyl group contains 8 to 22, and specifically about 10 to about 16 carbon atoms, and can be straight or branched chain, saturated or unsaturated. Examples include bis(2-hydroxyethyl) cocoamine oxide, bis(2-hydroxyethyl) tallow amine oxide, bis(2-hydroxyethyl) stearylamine oxide, and the like.

Additional class of useful alkamine oxide surfactants are termed alkamidopropyl di(lower alkyl) amine oxides in which the alkyl group contains 8 to 22, and specifically about 10 to about 16 carbon atoms, and can be straight or branched chain, saturated or unsaturated. Examples are cocoamidopropyl dimethyl amine oxide and tallowamidopropyl dimethyl amine oxide. Further useful alkamine oxide surfactants are termed alkylmorpholine oxides in which the alkyl group contains 8 to 22, and specifically about 10 to about 16, carbon atoms, and can be straight or branched chain, saturated or unsaturated.

The above classes of alkamine oxide surfactants contain a C₈-C₂₂ alkyl group selected from, for example, octyl, decyl, undecyl, lauryl, tridecyl, myristyl, cetyl, stearyl, isostearyl, oleyl, or a combination thereof.

Examples of alkamine oxide surfactants include decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germamidopropylamine oxide, isostearamido-propylamine oxide, stearamine oxide, stearamido-propylamine oxide, cocomorpholine oxide, decylamine oxide, dihydroxyethyl C₈-C₁₀ alkoxypropylamine oxide, dihydroxyethyl C₉-C₁₁ alkoxypropylamine oxide, dihydroxyethyl Ch-Che alkoxypropylamine oxide, dihydroxyethyl cocamine oxide, dihydroxyethyl stearamine oxide, dihydroxyethyl tallowamine oxide, hydrogenated tallow amine oxide, hydroxyethyl hydroxypropyl Cu-Cis alkoxypropylamine oxide, isostearamidopropyl morpholine oxide, myristamidopropylamine oxide, myristamine oxide, palmitamidopropylamine oxide, palmitamine oxide, PEG-3 lauramine oxide, tallow amidopropylamine oxide, tallow amine oxide, undecylenamidopropylamine oxide, lauryl/myristyl amidoamine oxide, or a combination thereof. Specific alkamine oxide surfactants are the alkyl di(lower alkyl) amine oxides in which the alkyl group contains about 12 to about 16 carbon atoms, including lauramine oxide, myristamine oxide, cocamine oxide, cetamine oxide, or a combination thereof.

In certain embodiments, the composition contains a combination of alkamine oxide surfactants comprising a first alkamine oxide containing 12 or fewer carbon atoms and a second alkamine oxide containing more than 12 carbon atoms.

Suitable anionic surfactants can include those anionic surfactants employed in skin cleansing formulations. Suitable anionic surfactants include, for example, alkyl sulfates, alkyl ether sulfates, alkyl aryl sulfonates, alpha-olefin sulfonates, alkali metal or ammonium slats of alkyl sulfates, alkali metal or ammonium salts of alkyl ether sulfates, alkyl phosphates, silicone phosphates, alkyl glyceryl sulfonates, alkyl sulfosuccinates, alkyl taurates, acyl taurates, alkyl sarcosinates, acyl sarcosinates, sulfoacetates, alkyl phosphate esters, mono alkyl succinates, monoalkyl maleates, sulfoacetates, alkyl phosphate esters, mono alkyl succinates, monoalkyl maleates, sulfoacetates, acyl isethionates, alkyl carboxylates, phosphate esters, sulfosuccinates (e.g., sodium dioctylsulfosuccinate), or a combination thereof. Specific examples of anionic surfactants include sodium lauryl sulfate, sodium lauryl ether sulfate, ammonium lauryl sulfosuccinate, ammonium lauryl sulfate, ammonium lauryl ether sulfate, sodium dodecylbenzene sulfonate, triethanolamine dodecylbenzene sulfonate, sodium cocoyl isethionate, sodium lauroyl isethionate, sodium N-lauryl sarcosinate, or a combination thereof. Specific anionic surfactants that are most useful include alkyl sulfates and alkyl ether sulfates. These alkyl and alkyl ether sulfate surfactants correspond to the general formula RO(C₂H₄O)ₓSO₃M wherein R is an alkyl or alkenyl group of from about 8 to about 30 carbon atoms: x is about 0 to about 10 on average, specifically about 1 to about 8, more specifically about 2 to about 4; and M is hydrogen or cation such as ammonium, alkanolammonium (e.g., triethanolammonium), a monovalent metal cation, such as sodium or potassium, or a polyvalent metal cation such as magnesium or calcium. M can be chosen so that the anionic surfactant is water soluble.

Nonionic surfactants have a hydrophobic domain and a hydrophilic domain. The hydrophilic domain lacks formal charges assigned to any of the associated groups in the hydrophilic domain The hydrophobic domain may be provided by a “fatty” region, often a hydrocarbon, for example, an alkyl chain. The hydrophilic region may be provided by a polyoxide. Polyglycols are a common hydrophilic domain due to their ability to incrementally adjust the properties of the nonionic surfactant by adjusting the length of the polyglycol. In some examples, the number of repeat units (generally, ethylene glycol or propylene glycol units) in a polyglycol is indicated by a number. For example, PEG-5 isononanate indicates five polyethylene glycol (PEG) groups attached to an isononanate residue.

Suitable nonionic surfactants include, for example, fatty alcohols, especially C₈ to C₂₂ fatty alcohols, which may be linear or branched, unsubstituted or substituted, saturated, unsaturated, or polyunsaturated. Examples of fatty alcohols include cetyl alcohol, stearyl alcohol, and myristyl alcohol Examples of substituted fatty alcohols include those having a polyglycol hydrophilic domain.

The type and amount of surfactant present in the composition is related to the identity of the surfactant or combination thereof, and the end use of the composition. The surfactant can be present in an amount of about 0.1 wt % to about 25 wt % based on the total weight of the antimicrobial cleaning composition, specifically about 1 wt % to about 20 wt %, more specifically about 5 wt % to about 15 wt %, and yet more specifically about 8 wt % to about 12 wt %.

The antimicrobial cleaning compositions may further comprise a thickener. Suitable thickeners include a polysaccharide such as a cellulose (e.g. cellulose, a cellulose derivative such as methyl cellulose, ethyl cellulose, a hydroxyalkyl cellulose (e.g., hydroxyethyl cellulose), a methyl hydroxyalkyl cellulose, a carboxy methyl cellulose, and the like), a fatty acid (mono) ethanolamide (e.g., cocamide monoethanolamine, and the like), an alginic acid or a physiologically acceptable salt thereof, agar agar, a starch fraction or starch derivative (e.g., amylose, amylopectin, dextrin), karaya gum, gellan gum, carob flour, gum arabic, dextran, guar gum, xanthan gum, a polyacrylate, a fatty acid ester of a methyl glucose ether, or a combination thereof.

When used, the thickener can be present in an amount of about 0.01 to about 30 wt %, specifically about 0.05 to about 20 wt % based on the total weight of the antimicrobial cleaning composition, and more specifically about 0.1 to about 10 wt %.

The antimicrobial cleaning compositions may further comprise a conditioning or moisturizing ester. Examples of such esters include cetyl myristate, cetyl myristoleate, and other cetyl esters, diisopropyl sebacate, and isopropyl myristate. In an embodiment, the ester is present in an amount of up to 10 wt % based upon the total weight of the antimicrobial cleaning composition, specifically about 0.5 to about 5 wt %, and more specifically about 1 to about 2 wt %.

Suitable salts include alkali and alkaline earth metal halides or alkali and alkaline earth metal salts with organic acids. Suitable salts include sodium chloride and potassium chloride.

Liquid cleaning compositions generally described herein comprise phytic acid, optionally a zinc compound, a liquid carrier including water or water and ethanol and glycol, and surfactant.

In an embodiment, a method of disinfecting comprises contacting a surface with an antimicrobial composition comprising phytic acid and a zinc compound, as described herein. In a further embodiment, the antimicrobial composition provides at least a 2-log, specifically at least a 3-log reduction of Staphylococcus aureus, Escherichia coli, Pseudomonas aeruginosa, or a combination thereof, when tested at a contact time of about 30 seconds according to ASTM E 2783. In another embodiment, the antimicrobial composition kills 99.9% bacteria based on a 15 to 30-second time point. Within these embodiments, the antimicrobial composition can be an antimicrobial cleaning composition, more specifically formulated as a liquid hand soap, body wash, or formulated as hand sanitizer or disinfecting wipes.

In an embodiment, a method of washing the skin of a human comprises washing the skin with an antimicrobial composition comprising phytic acid and a zinc compound, as described herein; and rinsing the antimicrobial composition from the skin. Within this embodiment, the washing with the antimicrobial composition provides at least a 2-log, specifically at least a 3-log reduction of Staphylococcus aureus, Escherichia coli, Pseudomonas aeruginosa, or a combination thereof, when tested at a contact time of about 15 to about 30 seconds according to ASTM E 2783. In a further embodiment, the antimicrobial composition is an antimicrobial cleaning composition as described herein, comprising phytic acid, a zinc compound, a surfactant, and a liquid carrier.

The following examples are merely illustrative of the use of phytic acid and compositions thereof disclosed herein and are not intended to limit the scope thereof.

### EXAMPLES

### Example 1. Study of Phytic Acid Versus Common Chelators for Rapid Time Kill Effects in Antibacterial Hand Soap Formulations

The formulas in Table 1 were batched using the same antibacterial hand soap base formulation to which the chelator is added, target pH of 4.8, and viscosity of 4500 cps. The % active content of phytic acid in Formula 3 was 0.045%. This value represents an equimolar quantity of phytic acid relative to the levels of EDTA and EDDS in Formulas 1 and 2, respectively.

TABLE 1

| | Formula 1 | Formula 2 | Formula 3 |
|---|---|---|---|
| | (Comparative, | (Comparative, | (Phytic acid) |
| Component | EDTA) Wt % | EDDS) Wt % | Wt % |
| EDTA | 0.05 | 0 | 0 |
| EDDS | 0 | 0.05 | 0 |
| Phytic acid (50% | 0 | 0 | 0.09 |
| phytic acid) | | | |

The following results were obtained using ASTM Standard E 2783 Standard Test Method for the Determination of Antimicrobial Activity for Water Miscible Compounds Using a Time Kill Procedure (ASTM E2783-11 (2016)). ASTM Standard E 2783 is used to assess the in vitro reduction of a microbial population of test organisms after exposure to a test material. The test method measures the changes of a population of aerobic and anaerobic microorganisms within a specific sampling time when tested against antimicrobial test materials in vitro. The organisms used are standardized as to growth requirements and inoculum preparation and must grow under the conditions of the test. The primary purpose of this test method is to provide a set of standardized conditions and test organisms to facilitate comparative assessments of antimicrobial materials miscible in aqueous systems. The organisms screened against in this study were selected to represent both Gram-negative and Gram-positive species.

TABLE 2

Staphylococcus aureus
ATCC 6538

| Formulation Tested | 15 Seconds | 30 Seconds |
|---|---|---|
| Formula 3 (Phytic acid) | 99.933% | 99.915% |
| Formula 2 (Comparative, EDDS) | 96.100% | 99.000% |
| Formula 1 (Comparative, EDTA) | 94.760% | 96.860% |

TABLE 3

Escherichia coli
ATCC 25922

| Formulation Tested | 15 Seconds | 30 Seconds |
|---|---|---|
| Formula 3 (Phytic acid) | >99.992% | >99.992% |
| Formula 2 (Comparative, EDDS) | 98.944% | 99.956% |
| Formula 1 (Comparative, EDTA) | >99.9999 | >99.9999 |

TABLE 4

Pseudomonas aeruginosa
ATCC 15442

| Formulation Tested | 15 Seconds | 30 Seconds |
|---|---|---|
| Formula 3 (Phytic acid) | 99.910% | 99.960% |
| Formula 2 (Comparative, EDDS) | 92.320% | 99.910% |
| Formula 1 (Comparative, EDTA) | 99.940% | 99.990% |

The results highlighted in BOLD demonstrate at least one additional factor of 10 (log-reduction) in the bacterial species count at that timepoint for phytic acid in comparison to the comparative formulation that uses EDDS. It has been observed, surprisingly, that phytic acid outperforms EDDS at both the 15-second and 30-second timepoints for rapid kill of S. aureus and E. coli. Additionally, it has been demonstrated that phytic acid outperforms EDDS at the 15-second time point for rapid kill of P. aeruginosa. What is more surprising, phytic acid has been found to outperform EDTA at both the 15-second and 30-second time points for rapid kill of S. aureus. Phytic acid was also found to perform comparably to EDTA (3-log reduction) at the 15-second time point for rapid kill of P. aeruginosa. Finally, the current product claim “Kills 99.9% bacteria” is based on the 30-second time point using EDDS, and it was demonstrated that phytic acid could support this claim by providing at least a 3-log reduction of each species after 30 seconds.

### Example 2. Study of Enhanced Phytic Acid-Containing Antibacterial Hand Soap Formulations

The formulas in the following tables were prepared by adding the indicated amount of benzalkonium chloride, phytic acid, and ZnSO₄ to a hand soap base formulation, target pH of 4.8, and viscosity of 4500 cps.

TABLE 5

| | Active % BKC | Active % Phytic | Active % |
|---|---|---|---|
| Formula ID | (Benzalkonium chloride) | acid | ZnSO₄ |
| A | 0.0000 | 0.2250 | 0.0000 |
| B | 0.1625 | 0.0000 | 0.0625 |
| C | 0.0406 | 0.0359 | 0.1484 |
| D | 0.0406 | 0.1484 | 0.0359 |
| E | 0.0000 | 0.0000 | 0.2250 |
| F | 0.1219 | 0.0359 | 0.0672 |
| G | 0.0812 | 0.0719 | 0.0719 |
| H | 0.1219 | 0.0672 | 0.0359 |
| I | 0.1625 | 0.0625 | 0.0000 |

TABLE 6

| | Mixture DOE | Mixture DOE | |
|---|---|---|---|
| Formula | Ratio BKC | Ratio Phytic | Mixture DOE |
| ID | (Benzalkonium chloride) | acid | Ratio ZnSO₄ |
| A | 0.0000 | 1.0000 | 0.0000 |
| B | 0.7222 | 0.0000 | 0.2778 |
| C | 0.1806 | 0.1597 | 0.6597 |
| D | 0.1806 | 0.6597 | 0.1597 |
| E | 0.0000 | 0.0000 | 1.0000 |
| F | 0.5417 | 0.1597 | 0.2986 |
| G | 0.3611 | 0.3195 | 0.3195 |
| H | 0.5417 | 0.2986 | 0.1597 |
| I | 0.7222 | 0.2778 | 0.0000 |

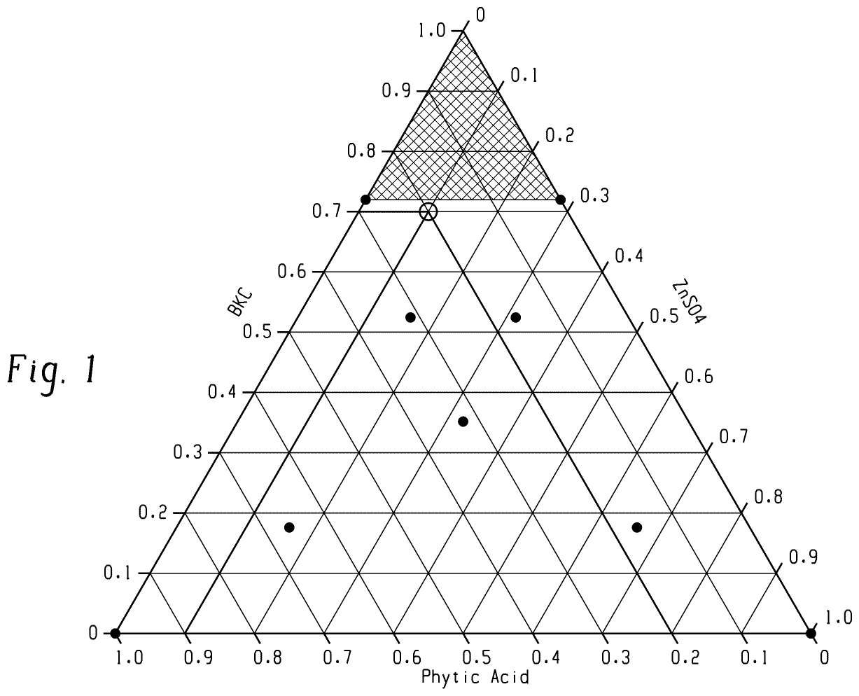

FIG. 1 illustrates a Ternary Plot showing the Mixture DOE design (the cursor was placed at an arbitrary point). Each solid black dot represents a unique combination of BKC/phytic acid/ZnSO₄ as outlined in the above tables. Each of the above compositions were screened against three bacteria species for rapid time kill at 30 seconds. The three species were E. coli (strain ATCC 25922), P. aeruginosa (strain ATCC 15442), and S. aureus (strain ATCC 6538). These organisms were selected to represent both Gram-negative and Gram-positive species. The following results were obtained using ASTM Standard E 2783 Standard Test Method for the Determination of Antimicrobial Activity for Water Miscible Compounds Using a Time Kill Procedure.

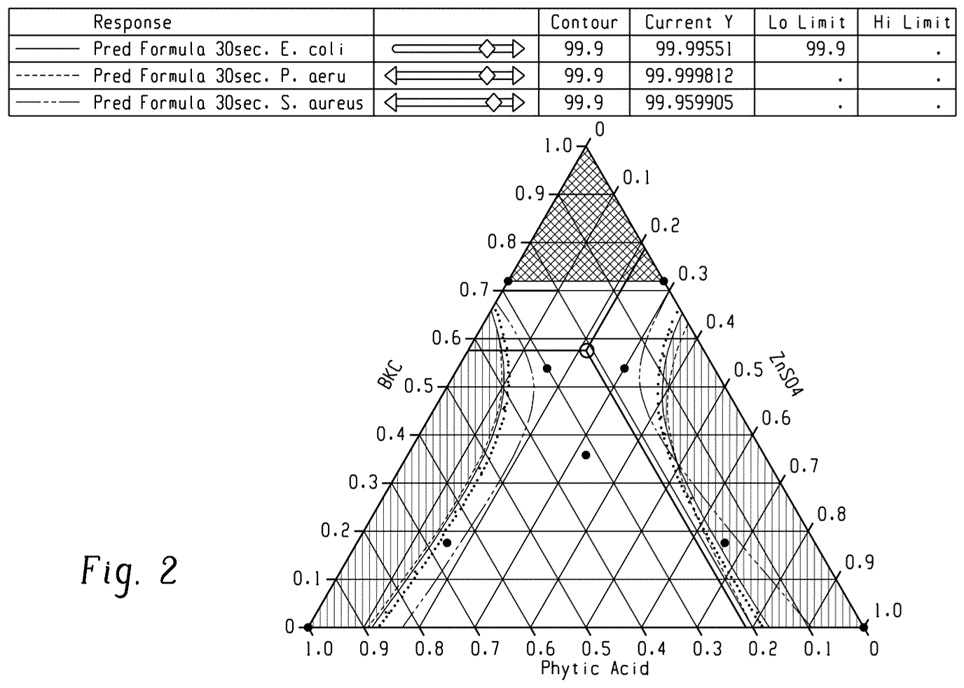

Efficacy against E. coli (strain ATCC 25922) is shown in a mixture profiler which has set a minimum time kill efficacy threshold of 99.9% (FIG. 2). The output from the profiler shows compositions expected to have <99.9% efficacy (vertical line-shaded regions) and compositions that expect to have at least 99.9% efficacy (white region). In a Summary of Fit, the resulting R² value of ˜92.5% for the E. coli model was highly satisfactory. This result suggests that ˜92.5% of the variability in the E. coli time kill response can be explained by the model. This is quite good considering the dynamic nature of microorganisms.

The Effects Summary, Analysis of Variance (ANOVA), and Parameter Estimates data demonstrate that the overall model and each individual factor (BKC/Phytic acid/ZnSO₄) were found to be statistically significant (p-values<0.05). A surprising and unexpected result can be observed in the statistical significance of the ‘Phytic acid*ZnSO₄’ two-way interaction. A positive ˜4.266 coefficient for this interaction in the Parameter Estimates suggests that there is a synergistic effect occurring between phytic acid and ZnSO₄. Without being bound by theory, it is possible that the phytic acid, being a strong chelator, is forming a complex in situ (in the neat formulation) with free Zn²⁺ atoms. This complex may be serving as a delivery system for the Zn²⁺ atoms to more effectively reach the bacterial cell membrane to perform as a bactericidal agent. If this coefficient was found to be negative, it would demonstrate that this in situ interaction would be making both the phytic acid and Zn²⁺ less freely available to interact with the bacteria, yet this antagonistic and inhibitive effect was not observed.

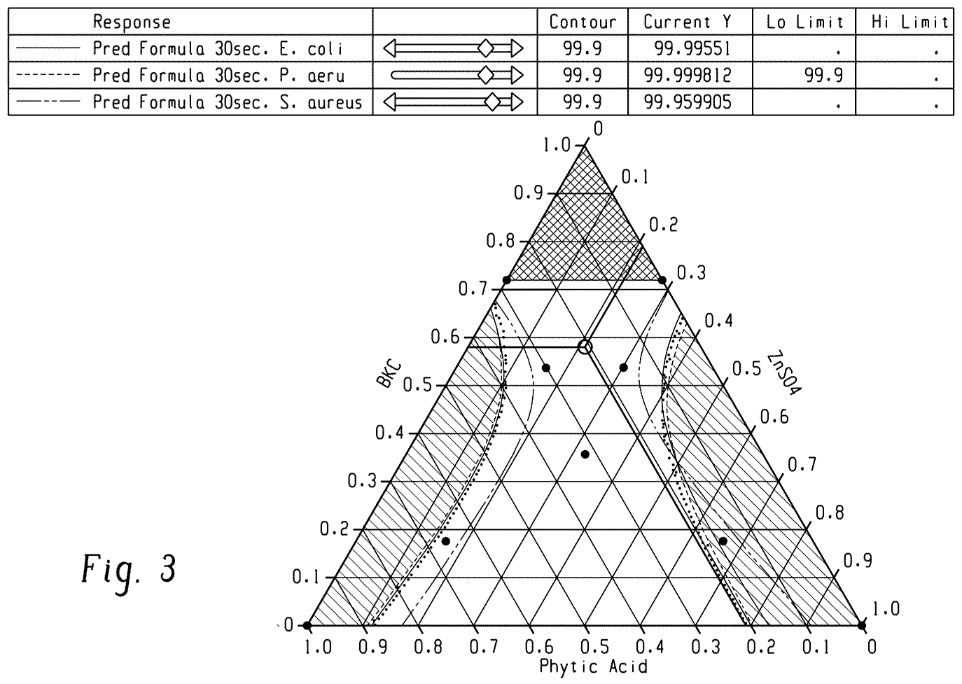

Efficacy against P. aeruginosa (strain ATCC 15442) is shown in a mixture profiler which has set a minimum time kill efficacy threshold of 99.9% (FIG. 3). The output from the profiler shows compositions expected to have <99.9% efficacy (diagonal line-shaded regions) and compositions that expect to have at least 99.9% efficacy (white region).

The results observed for P. aeruginosa are similar to those observed and explained above for E. coli. The model and individual factors were all found to be statistically similar (p-values<0.05). Once again, a surprising and unexpected synergistic benefit was observed through the statistical significance of the ‘Phytic acid*ZnSO4’ two-way interaction with a positive coefficient of ˜3.95.

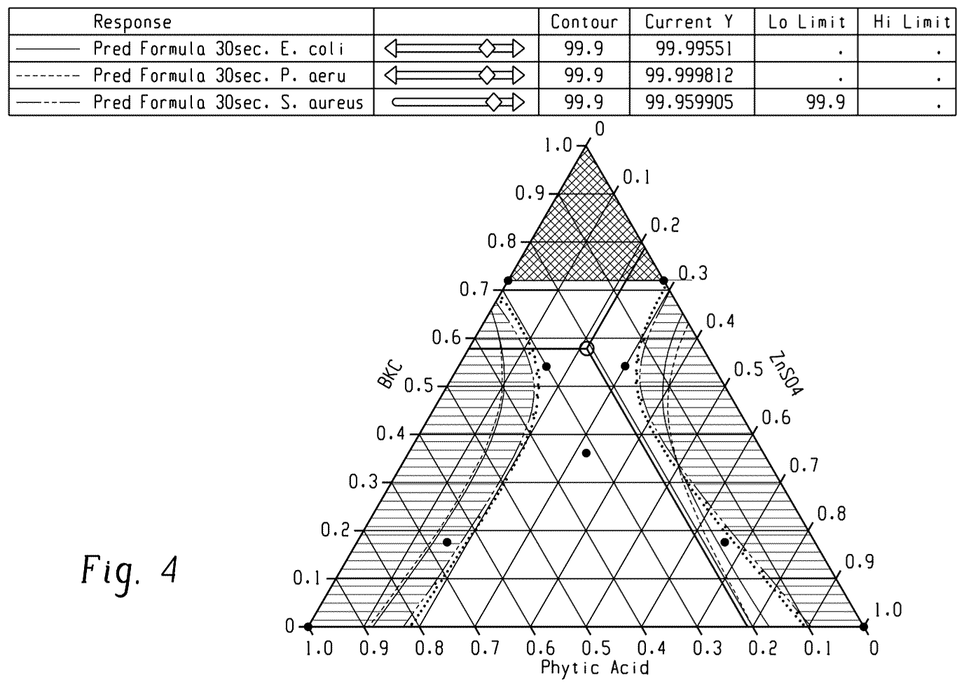

Efficacy against S. aureus (strain ATCC 6538) is shown in a mixture profiler which has set a minimum time kill efficacy threshold of 99.9% (FIG. 4). The output from the profiler shows compositions expected to have <99.9% efficacy (horizontal line-shaded regions) and compositions that expect to have at least 99.9% efficacy (white region).

The results observed for S. aureus are similar to those observed and explained above for E. coli and P. aeruginosa. The model and individual factors were all found to be statistically similar (p-values<0.05). Once again, a surprising and unexpected synergistic benefit was observed through the statistical significance of the ‘Phytic acid*ZnSO4’ two-way interaction with a positive coefficient of ˜5.25.

TABLE 7

| Effect Summary | E. coli | P. aeruginosa | S. aureus |
|---|---|---|---|
| Source | PValue | PValue | PValue |
| Phytic acid | 0.00000 | 0.00000 | 0.00000 |
| ZnSO4 | 0.00000 | 0.00000 | 0.00000 |
| BKC | 0.00000 | 0.00000 | 0.00000 |
| Phytic acid*ZnSO4 | 0.00017 | 0.00000 | 0.00228 |
| BKC*Phytic acid | 0.27184 | 0.05945 | 0.23599 |
| BKC*ZnSO4 | 0.52305 | 0.65306 | 0.53214 |

| Parameter Estimates | E. coli | | P. aeruginosa | | S. aureus | |
|---|---|---|---|---|---|---|
| Term | Estimate | Std Error | Estimate | Std Error | Estimate | Std Error |
| Phytic acid | 99.492528 | 0.046331 | 99.560215 | 0.026586 | 99.502864 | 0.075857 |
| ZnSO4 | 99.264549 | 0.046331 | 99.204845 | 0.026586 | 99.08368 | 0.075857 |
| BKC | 100.41947 | 0.24672 | 100.38312 | 0.140929 | 100.55469 | 0.402112 |
| Phytic acid*ZnSO4 | 4.2665989 | 0.924353 | 3.9545144 | 0.530745 | 5.257756 | 1.514376 |
| BKC*Phytic acid | −0.908691 | 0.804131 | −0.915 | 0.459171 | −1.59839 | 1.310155 |
| BKC*ZnSO4 | −0.522734 | 0.804131 | −0.209387 | 0.459171 | −0.832252 | 1.310155 |

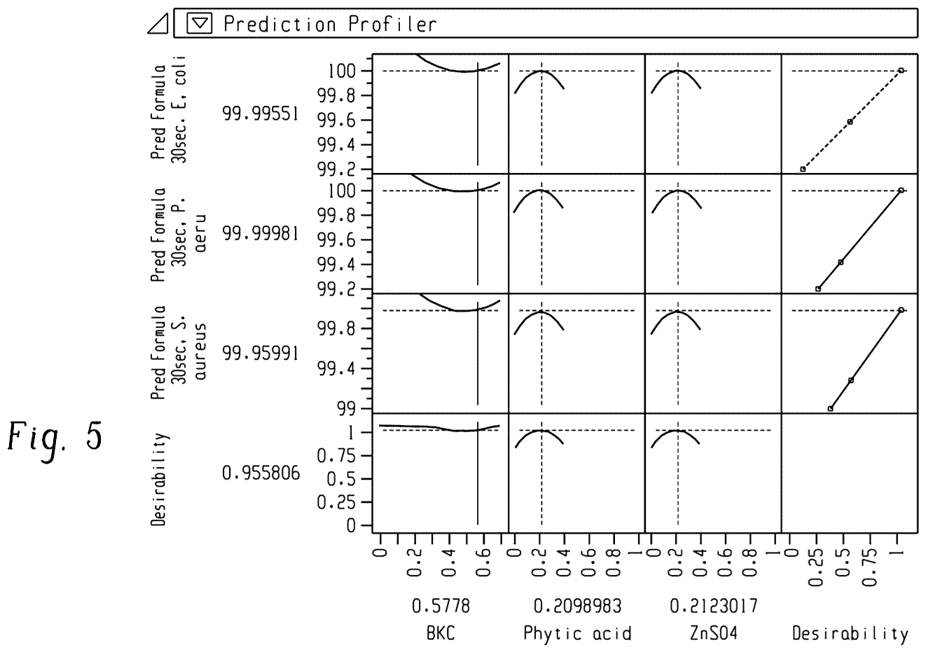

A prediction profiler (FIG. 5) was created to identify compositions which maximize the desirability of the overall model. In this case, “maximized desirability” translates to highest predicted time kill efficacy. For this model, the proportion of BKC was fixed at 0.5778 prior to maximizing the desirability. This proportion represents 0.13% BKC in a final formulation. In an embodiment, a liquid hand soap comprises phytic acid, zinc sulfate, and water, wherein each of the phytic acid and zinc sulfate independently is in an amount of about 0.001 wt % to about 0.1 wt %, and specifically about 0.01 wt % to about 0.07 wt % based on the total weight of the liquid hand soap. In another embodiment, the liquid hand soap further comprises benzalkonium chloride and an amphoteric surfactant, including an alkamine oxide.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “+10%” means that the indicated measurement can be from an amount that is minus 10% to an amount that is plus 10% of the stated value. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. An antimicrobial composition, comprising:
phytic acid;
a zinc compound; and
a liquid carrier.

2. The composition of claim 1, wherein phytic acid and the zinc compound are in a weight ratio of about 75:25 to about 25:75.

3. The composition of claim 1, wherein phytic acid and the zinc compound are in a weight ratio of about 60:40 to about 40:60.

4. The composition of claim 1, wherein the zinc compound is zinc sulfate, zinc picolinate, zinc citrate, zinc acetate, zinc glycerate, zinc gluconate, zinc monomethionine, zinc chloride, zinc iodide, zinc oxide, or a combination thereof.

5. The composition of claim 1, wherein the zinc compound is zinc sulfate.

6. The composition of claim 1, in the form of an antimicrobial cleaning composition further comprising a surfactant.

7. The composition of claim 6, wherein the surfactant is a cationic surfactant, an amphoteric surfactant, a nonionic surfactant, or a combination thereof.

8. The composition of claim 6, wherein the surfactant is an amphoteric surfactant.

9. The composition of claim 6, further comprising an additional biocide, a buffer, a colorant/dye, a fragrance, a humectant, an emollient, a natural plant oil or extract, a pH adjusting agent, a preservative, a salt, a thickener, or a combination thereof.

10. The composition of claim 1, wherein the liquid carrier is an aqueous carrier comprising water and optionally ethanol, propanol, isopropanol, propylene glycol, glycerin, or a combination thereof.

11. The composition of claim 1, wherein the composition has a pH of about 3.5 to about 7.6, specifically about 4.0 to about 7.0, more specifically about 4.5 to about 6.0, yet more specifically about 4.6 to about 5.5, and still yet more specifically about 4.7 to about 5.0.

12. The composition of claim 1, formulated as a liquid hand soap, body wash, disinfecting wipe, cleaning solution, or hand sanitizer.

13. The composition of claim 6, formulated as a liquid hand soap.

14. The composition of claim 13, comprising
phytic acid in an amount of about 0.001 wt % to about 2.0 wt %;
a zinc compound in an amount of about 0.001 wt % to about 2.0 wt %; and
a surfactant in an amount of amount of about 0.1 wt % to about 25 wt % based on the total weight of the composition.

15. The composition of claim 14, wherein the zinc compound is zinc sulfate.

16. The composition of claim 14, further comprising benzalkonium chloride.

17. A method of disinfecting, comprising:
contacting a surface with an antimicrobial composition comprising phytic acid and a zinc compound.

18. The method of claim 17, wherein the antimicrobial composition:
provides at least a 2-log, specifically at least a 3-log reduction of Staphylococcus aureus, Escherichia coli, Pseudomonas aeruginosa, or a combination thereof when tested at a contact time of about 30 seconds according to ASTM E 2783; or
kills 99.9% bacteria based on a 15-second or a 30-second time point.

19. The method of claim 18, wherein the antimicrobial composition is a consumer product, specifically a liquid hand soap.

20. A method of washing the skin of a human, comprising:
washing the skin with an antimicrobial cleaning composition; and
rinsing the antimicrobial cleaning composition from the skin;
wherein the antimicrobial cleaning composition comprises phytic acid, a zinc compound, a surfactant, and a liquid carrier.
